(19) United States
(12) Patent Application Publication
(10) Pub. No.: US 2007/0239194 A1
(43) Pub. Date: Oct. 11, 2007

(54) Vaso-occlusive devices having expandable fibers

(75) Inventors: Tri D. Tran, Fremont, CA (US); Mediko Issakhani, San Jose, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 11/400,595

(22) Filed: Apr. 5, 2006

(51) Int. Cl. A61M 29/00

(52) U.S. Cl. 606/191; 606/200

(57) ABSTRACT

This is a device for occluding a space, for example an aneurysm, within the body. In particular, the device comprising a metallic vaso-occlusive device and expandable fibrous elements. The devices may be placed in a desired site within a mammal.

### FIELD OF THE INVENTION

Compositions and methods for repair of aneurysms are described. In particular, vaso-occlusive devices comprising shaped metallic device with attached expansile fibers are described.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel coils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel.

One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids. See, e.g., U.S. Pat. No. 6,299,627. Vaso-occlusive coils having attached fibrous elements are disclosed in U.S. Pat. No. 5,833,705 to Ken as well as U.S. Pat. No. 5,304,194 to Chee.

U.S. Pat. No. 5,354,295 and its parent, U.S. Pat. No. 5,122,136, both to Guglielmi et al., describe an electrolytically detachable embolic device. Vaso-occlusive coils having little or no inherent secondary shape have also been described. For instance, co-owned U.S. Pat. Nos. 5,690,666; 5,826,587; and 6,458,119 by Berenstein et al., describes coils having little or no shape after introduction into the vascular space. U.S. Pat. No. 5,382,259 describes non-expanding braids covering a primary coil structure.

Vaso-occlusive compositions comprising one or more expandable hydrogels have also been described. See, e.g., U.S. Pat. Nos. 6,960,617 and 6,113,629. U.S. Pat. Nos. 6,602,261 and 6,238,403 discloses a plurality of expansible hydrogel elements disposed at spaced intervals along a filamentous carrier. U.S. Pat. Nos. 6,616,617; 6,475,169; 6,168,570 and 6,159,165 disclose multi-stranded microcable devices, where one or more of the stands may be an expandable material.

However, there remains a need for vaso-occlusive devices that combine the advantages of metallic vaso-occlusive devices (e.g., coils) and expansile materials (e.g., hydrogels) in a single vaso-occlusive device.

### SUMMARY

Thus, this invention includes novel occlusive devices as well as methods of using and making these devices.

In one aspect, the invention includes a vaso-occlusive device comprising a metallic vaso-occlusive device; and one or more fibrous elements, wherein at least one of the fibrous elements comprises an expandable material. In certain embodiments, the fibrous elements comprising an expandable material comprise a polymer (e.g., PET) coated or permeated with the expandable material. In other embodiments, the expandable material comprises a hydrogel material.

In any of the devices described herein, the fibrous elements may be monofilaments or multifilaments. In certain embodiments, the fibrous elements comprise at least one monofilament. Multiple monofilaments may be structured over the vaso-occlusive device, for example to form an open-weave structure surrounding the metallic vaso-occlusive device.

Furthermore, in any of the devices described herein, the fibrous elements may be attached to the metallic vaso-occlusive device at one or more locations.

The metallic vaso-occlusive device may comprise any metal, for example a metal selected from the group consisting of nickel, titanium, platinum, gold, tungsten, iridium and alloys or combinations thereof. In certain embodiments, the metallic vaso-occlusive device comprises nitinol and/or platinum.

In certain embodiments, the metallic vaso-occlusive device comprises a coil shape. In other embodiments, the metallic vaso-occlusive device comprises a tubular braid.

Any of the devices described herein may further comprise one or more additional materials, for example, at least one bioactive material. Any of the devices described herein may further comprise a severable junction detachably which may be connected to a pusher element. The detachment junction can be positioned anywhere on the device, for example at one or both ends of the device. In certain embodiments, the severable junction(s) are, an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof. The detachment junction(s) may be attached to one or more expansile fibers material or, preferably, to one or more vaso-occlusive devices.

In another aspect, a method of occluding a body cavity is described, the method comprising introducing any of the devices as described herein into the body cavity. In certain embodiments, the body cavity is an aneurysm.

These and other embodiments of the subject invention will readily occur to those of skill in the art in light of the disclosure herein.

### BRIEF DESCRIPTION OF THE FIGURES

FIG. 1 is a side view of an exemplary device as described herein. The device comprises a metallic vaso-occlusive coil 10 and expansible fibrous elements 12. This embodiment shows a device having multiple expandable monofilaments 12 attached to various winds of the metallic vaso-occlusive coil 10. FIG. 1 shows the device when the fibers 12 are not expandable.

FIG. 2 is a side view of the exemplary device shown in FIG. 1 and shows the device after expansion of the fibrous elements 12.

FIG. 3 is a side view of another exemplary device as described herein. The device comprises a metallic vaso-occlusive coil 20 and expandable fibrous elements 22. This embodiment has at least one expansible monofilament fiber 22 attached to every wind of the metallic vaso-occlusive coil 20. FIG. 3 shows the device when the expandable material of the monofilament fibers 22 is not expanded.

FIG. 4 is a side view of the exemplary device shown in FIG. 3 and shows the device after expansion of the expandable materials of the fibers 22.

FIG. 5 is a side view of another exemplary device as described herein. The device comprises a metallic vaso-occlusive coil 30 and expansible fibrous elements 32. The expansible fibrous elements 32 form an open-woven lattice over the metallic vaso-occlusive coil 30. FIG. 5 shows the device when the expandable fibers 32 are not expanded.

FIG. 6 is a side view of the exemplary device shown in FIG. 5 and shows the device after expansion of the fibrous elements 32.

FIG. 7 is a side view of another exemplary device as described herein. The device comprises a metallic vaso-occlusive coil 44 and an expandable monofilament 42 wrapped loosely around the metallic coil 44.

FIG. 8 is a side view of another exemplary device as described herein. The device comprises a metallic coil 44 and two expandable monofilaments 42, 43 wrapped loosely around the metallic coil 44.

It is to be understood that the drawing depicts only exemplary embodiments and are not to be considered limiting in scope.

### DETAILED DESCRIPTION

Occlusive (e.g., embolic) devices are described. The implantable devices described herein combine the advantages of platinum coils (e.g., enhanced tissue growth and healing) with the advantages of expandable materials (e.g., space-filling). Methods of making and using these vaso-occlusive devices also form aspects of this invention.

All documents (publications, patents and patent applications) cited herein, whether above or below, are hereby incorporated by reference in their entireties.

It must be noted that, as used in this specification and the appended claims, the singular forms “a”, “an”, and “the” include plural references unless the content clearly dictates otherwise. Thus, for example, reference to an implant comprising “an expandable fiber” includes implants comprising of two or more of such fibers.

The implantable devices described herein comprise one or more expandable fibers. The terms “expansile,” “expandable,” and “expansible” are used interchangeable to refer to any material that is capable of expansion. The material may self-expandable and/or expand upon exposure to one or more stimuli (water, light, heat, etc.).

In certain embodiments, the expansible material comprises a hydrogel that expands upon contact with water. Hydrogels may be biodegradable and/or have regulated expansion. Non-crosslinked hydrogels are described, for example, in U.S. patent application Ser. No. 11/242,981, filed Oct. 4, 2005, entitled “Self-Expandable Coil is Regulated Expansion,” incorporated by reference herein in its entirety.

Hydrogels are commercially available and well known those of skill in the art. See, e.g., U.S. Pat. Nos. 6,818,018; 6,602,261; 6,238,403; 6,245,090; 5,823,198; 5,570,585; 5,456,693; 5,258,042; and 4,663,358, describing water-swellable crosslinked hydrogels and porous hydrated polyvinyl alcohol (PVA) foam gels). Expanding hydrogels can also include, by way of example and not by way of limitation, gels formed from polysaccharides and mucopolysaccharides including, but not limited to hyaluronates, pectins, agarose, alginate; chitosan, chitosan derivatives such as chitosan modified with fructose, galactose and/or proteins such as collagen, gelatin and albumin; gels formed from proteins such as collagen, gelatin, fibronectin, fibrin, albumin, or poly or copolypeptides; carboxy alkyl celluloses, including but not limited to carboxymethyl cellulose; partially oxidized cellulose; and gels formed from synthetic biodegradable polymers such polyphosphazenes, polyphosphoesters, polyanhydrides, polyethylene oxides, polyethylene oxide-co-polypropyleneoxide block copolymers, polylactides, polyglycolide, polycaprolactone, poly(3-hydroxy-butyric acid), polyvinyl alcohols, PEG, dextran, alginic acid and sodium alginate and others such as described in U.S. Pat. No. 4,526,938 to Chirchill, et al.; gels formed from other hydroxy acids; and/or gels formed from other biologically degradable polymers that are non-toxic or are present as metabolites in the body.

Furthermore, expansion of the hydrogels may be regulated by including a polymer composition such as the PEO/PLA or PLA-PGA-PEO ratio and/or the length of the PLA-PEO blocks in the PEO-PLA or PLA-PGA-PEO block copolymers (see, e.g., Younes et al. (1987) J. Biomed. Mater Res 21(11):1301-1306;) Younes et al. (1988) Biomater Artif Cells Artif Organs. 16(4):705-19) or by the density of physical cross-linking achieved by variation of Polymer/Physical cross-linker such as Polymer/Ca ion ratio in Ca-alginate gels.

Inorganic expansible materials can also be derived from, by way of example and not by way of limitation, silicones, alumina, and ferric oxide.

Expandable materials having increased macroporosity are described, for example, in U.S. patent application Ser. No. 11/051,578, filed Feb. 4, 2005, entitled “Macroporous Materials for Use in Aneurysms,” and U.S. patent application Ser. No. 11/347,080, filed Feb. 2, 2006, entitled “Porous Materials to Enhance Wound Healing in the Aneurysm,” both of which documents are incorporated by reference herein in their entireties.

Aneurysms treated with metallic (e.g., platinum) vaso-occlusive coils have been shown to have enhanced tissue growth inside the aneurysm as compared to aneurysms treated with non-metallic devices (e.g., hydrogels). Enhanced tissue growth promotes faster healing and, in addition, reduces the formation of scar tissue. Reducing scar tissue in turn renders the occluded vessel less susceptible to recanalization. Thus, the vaso-occlusive devices described herein combine the advantages of the healing properties of metallic devices with the advantageous volume (space) filling properties of expandable materials, thereby providing devices that reduce the rate of recanalization in aneurysms, particularly wide neck, large-sized and/or bifurcated aneurysms.

Thus, the devices described herein include at least one metallic vaso-occlusive device. Although depicted in the Figures as a coil, it will be apparent that the metallic vaso-occlusive device may be of a variety of shapes or configuration including, but not limited to, braids, wires, tubes (e.g., perforated or slotted tubes), injection-molded devices, knits and/or woven structures that include at least one metal and the like. See, e.g., U.S. Pat. No. 6,533,801 and International Patent Publication WO 02/096273.

The metallic vaso-occlusive device comprise one or more metals or metal alloys, for example, Platinum Group metals, especially platinum, rhodium, palladium, rhenium, as well as tungsten, gold, silver, tantalum, stainless steel and alloys of these metals. Particularly preferred is platinum. In certain embodiments, the metallic device maintains its shape despite being subjected to high stress, for example, “super-elastic alloys” such as nickel/titanium alloys (48-58 atomic % nickel and optionally containing modest amounts of iron, also known as nitinol); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). See, e.g., U.S. Pat. Nos. 3,174,851; 3,351,463; and 3,753,700.

The metallic vaso-occlusive devices described herein may change shape upon release from the restraining member, for example change from a constrained linear form to a relaxed, three-dimensional configuration upon deployment.

In addition to the metallic vaso-occlusive device, the devices described herein also include at least one expansible material. The expandable material can be combined with a metallic device in any suitable way. For example, in certain embodiments, the expansible material is coated directly onto the metallic device (e.g., coil) such that at least some of the metal remains exposed. Alternatively, the metallic coil and expandable materials can be delivered separately (in an order) to the aneurysm.

In certain preferred embodiments, one or more carriers (e.g., fibrous elements) comprising the expandable material(s) is(are) combined with a metallic device. It will be apparent that the carrier can have any dimensions so long as when it is combined with the metallic device, the metal of the metallic device is at least partially exposed. Furthermore, a single device may include carriers of different dimensions. As depicted in the drawings, the carrier for the expansible material(s) can be a fibrous element comprising one or more fibers.

It will also be apparent that any number of carriers comprising expandable materials can be used on a single device. The spacing and/or dimensions of the carriers may be regular or irregular. In addition, the device may include one or more additional filamentous materials that are not expandable.

When a carrier (e.g., fiber) comprising one or more expandable materials is used, the carrier may be coated or permeated with one or more expandable materials before or after being combined with the metallic vaso-occlusive device. Methods of coating and/or permeating fibrous elements with expandable materials such as hydrogels are known in the art. It is not required that every carrier (e.g., fibrous element) comprise expandable material(s). In addition, it will readily apparent that different carriers (e.g., fibrous elements) can comprise different expandable materials or combinations of expandable materials.

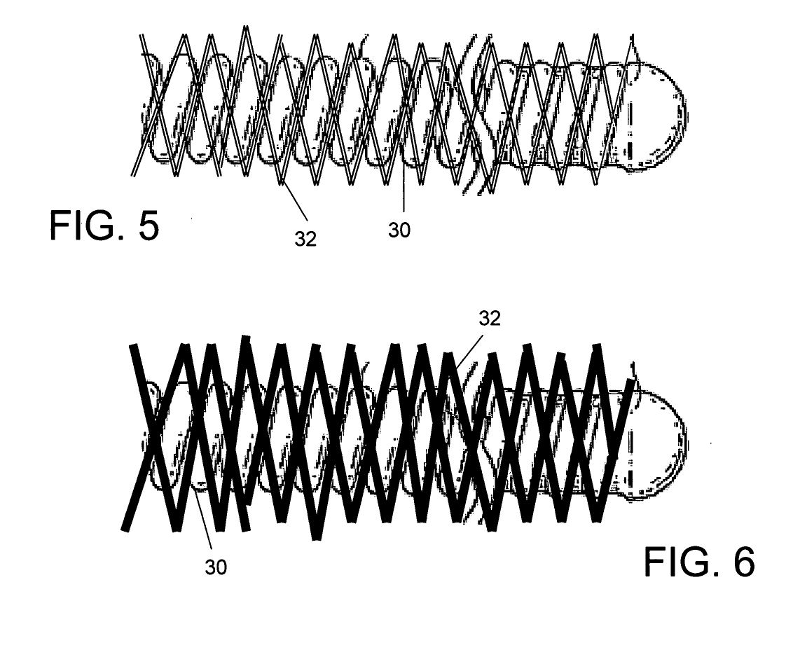

As noted above, the carrier (e.g., fibrous element) may be combined to the metallic device by any suitable means, including, but not limited to, winding (threading) of the fibers through the device, knotting the fibers at one more locations (e.g., knotting the ends to the device), gluing, etc. See, also, U.S. Pat. Nos. 5,935,145; 5,833,705; 5,549,624; 5,522,822; 5,382,259; 5,304,194; and 5,226,911. The specific location(s) of contact (and/or attachment) is(are) not critical. The carrier (e.g., fibrous element) may make a pattern (e.g., open woven structure as shown in FIGS. 5 and 6). Furthermore, the fibrous element(s) may be fully or partially exterior and/or interior to the metallic device.

The carrier (e.g., fiber) may be made from a variety of materials, including but not limited to metals, polymers and combinations thereof, including but not limited to, stainless steel, platinum, kevlar, carbothane, cyanoacrylate, epoxy, poly(ethyleneterephthalate) (PET), polytetrafluoroethylene (Teflon™), polypropylene, polyimide polyethylene, polyglycolic acid, polylactic acid, nylon, polyester, fluoropolymer, and copolymers or combinations thereof. See, e.g., U.S. Pat. Nos. 6,585,754 and 6,280,457 for a description of various polymers. Particularly preferred is a polymer such as PET. Any one device as described herein may include multiple carriers (e.g., fibers) comprising different materials.

The carrier(s) coated and/or permeated with the expandable material may include one or more bundles of individual fibers (e.g., 5 to 100 fibers per bundle, preferably 20 to 30 fibers per bundle) or one or more monofilaments. Furthermore, one or more bundles and/or one or more monofilaments may be combined with the same device.

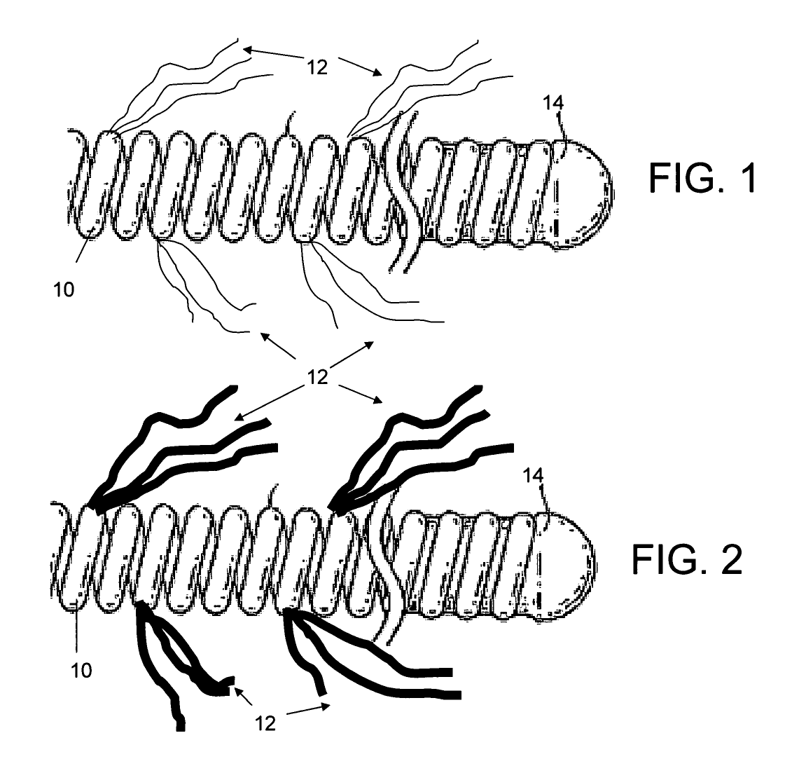

FIG. 1 depicts an exemplary embodiment of the devices as described herein and depicts a design in which multiple monofilaments comprising expansible materials 12 are spaced randomly throughout the metallic coil 10. As depicted, multiple monofilaments are attached to the same winds of the coil. It will be apparent that the number of monofilaments attached to particular wind of a coil can vary greatly and that different numbers of fibrous elements may be attached to any, some or all of the metallic vaso-occlusive coil. Also shown in FIG. 1 is optional end tip 14. FIG. 1 shows the device prior to expansion.

FIG. 2 shows the device of FIG. 1 following expansion of the expansible fibrous elements. The overall volume taken up by the device is enhanced.

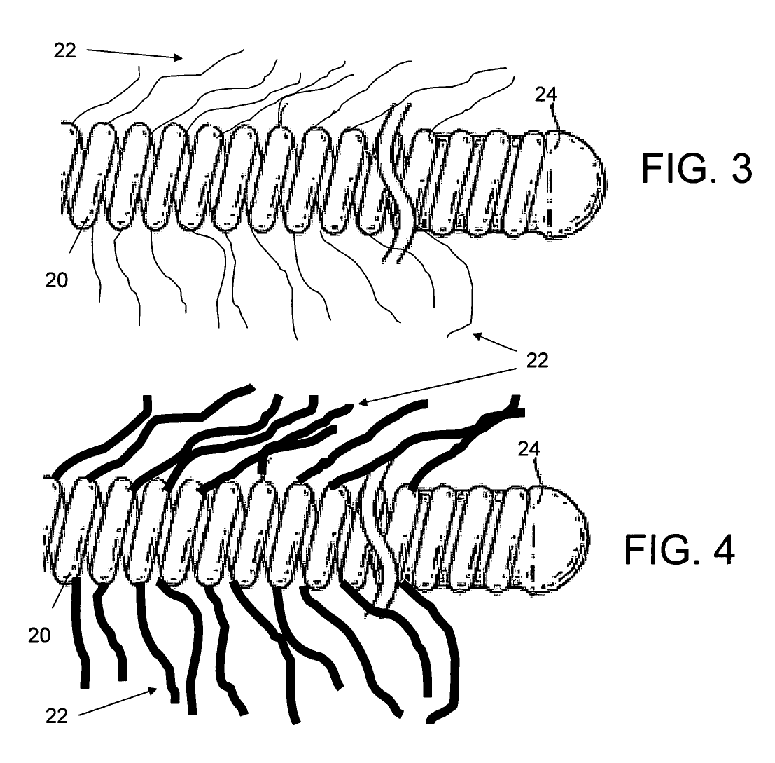

FIG. 3 depicts another exemplary embodiment in which a single expandable filament 22 is attached at every wind of the metallic coil 20. Optional end cap 24 is also shown. FIG. 3 shows the device prior to expansion.

FIG. 4 shows the device of FIG. 3 after expansion of the expansible fiber.

FIG. 5 depicts yet another embodiment in which expandable fibers 32 are braided in an open-weave configuration around a metallic coil 30. FIG. 5 shows the device prior to expansion of the expandable materials and FIG. 6 shows the device after expansion of these materials.

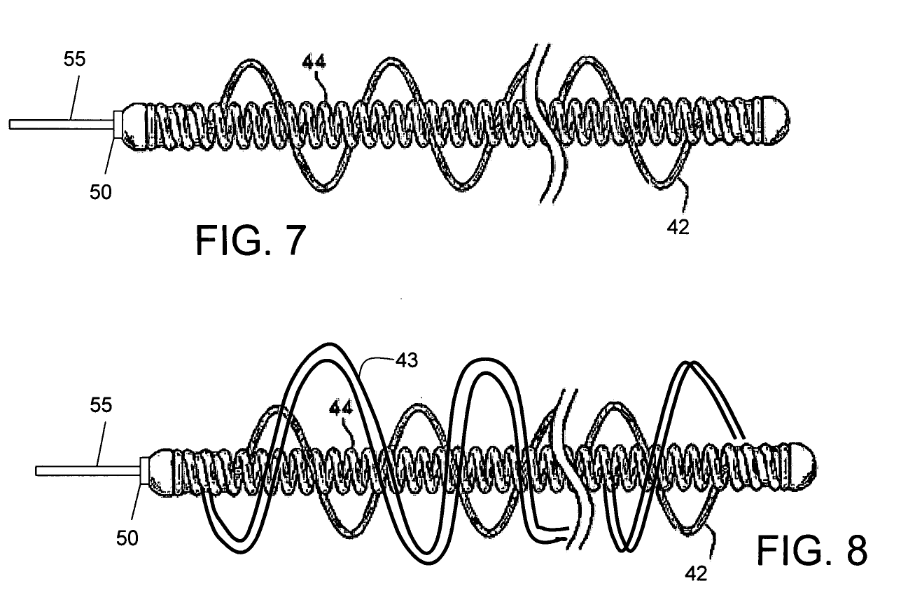

FIG. 7 shows yet another embodiment in which a single fiber comprising at least one expandable material 42 is loosely wrapped around a metallic coil 44.

FIG. 8 shows another embodiment in which two strands of fibers, each fiber comprising at least one expandable material 42, 43, are loosely wrapped around a metallic coil 44.

As shown in FIGS. 7 and 8, any of the devices described herein may further comprise a detachment junction 50, which is severable. See, e.g., U.S. Pat. Nos. 5,354,295 and 5,122,136. The detachment junction may be connected to a pusher element, such as a pusher wire 55. The detachment junction can be positioned anywhere on the device, for example at one or both ends of device.

The severable junction(s) may be detached in a variety of ways, for example using an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof. Furthermore, the detachment mechanism may be hydraulic, for example the pusher wire may be cannulated, for example to allow for saline injection through the pusher wire to push off the coil.

The devices described herein may also comprise further additional components, such as co-solvents, plasticizers, coalescing solvents, bioactive agents, antimicrobial agents, porogens, antithrombogenic agents (e.g., heparin), antibiotics, pigments, radiopacifiers and/or ion conductors which may be coated using any suitable method or may be incorporated into the element(s) during production. See, e.g., co-owned U.S. Patent Application Publication No. 20050149109, published Jul. 7, 2005; U.S. Pat. No. 6,585,754 and WO 02/051460, incorporated by reference in their entireties herein. The bioactive materials can be coated onto the device (e.g., anticoagulants, growth factors, extracellular matrix components, living cells, DNA fragments, clotting stabilizers, or other materials intended to enhance or encourage wound healing) and/or can be placed in the vessel prior to, concurrently or after placement of one or more devices as described herein.

As noted above, one of more of the elements (e.g., metallic vaso-occlusive device, fibers comprising expandable material, additional materials) may also be secured to each other or to the device at one or more locations. For example, to the extent that various elements are thermoplastic, they may be melted or fused to other elements of the devices. Alternatively, they may be glued or otherwise fastened. Furthermore, the various elements may be secured to each other in one or more locations.

Methods of Use

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the vaso-occlusive device at the distal end, is advanced through the catheter.

Once the selected site has been reached, the vaso-occlusive device is extruded, for example by loading onto a pusher wire. Preferably, the vaso-occlusive device is loaded onto the pusher wire via a mechanically or electrolytically cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. Nos. 6,623,493 and 6,533,801 and International Patent publication WO 02/45596. They are held in place by gravity, shape, size, volume, magnetic field or combinations thereof.

It will also be apparent that the operator can remove or reposition (distally or proximally) the device. For instance, the operator may choose to insert a device as described herein, before detachment, move the pusher wire to place the device in the desired location.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device comprising
a metallic vaso-occlusive device; and
one or more fibrous elements, wherein at least one of the fibrous elements comprises an expandable material.

2. The device of claim 1, wherein the fibrous elements comprising an expandable material comprise a polymer coated or permeated with the expandable material.

3. The device of claim 2, wherein the polymer comprises PET.

4. The device of claim 1, wherein the expandable material comprises a hydrogel material.

5. The device of claim 1, wherein the fibrous element comprises at least one monofilament.

6. The device of claim 5, wherein the monofilaments form an open-weave structure surrounding the metallic vaso-occlusive device.

7. The device of claim 1, wherein the fibrous element comprises at least one multifilament.

8. The device of claim 1, wherein the fibrous elements are attached to the metallic vaso-occlusive device at one or more locations.

9. The device of claim 1, wherein the metallic vaso-occlusive device comprises a metal is selected from the group consisting of nickel, titanium, platinum, gold, tungsten, iridium and alloys or combinations thereof.

10. The device of claim 9, wherein the metal is nitinol or platinum.

11. The device of claim 10, wherein the metal is platinum.

12. The device of claim 1, wherein the metallic vaso-occlusive device comprises a coil.

13. The device of claim 1, wherein the metallic vaso-occlusive device comprises a tubular braid.

14. The device of claim 1, wherein the device further comprises an additional material.

15. The device of claim 14, wherein the additional material comprises at least one bioactive material.

16. The device of claim 1, wherein the device further comprises a detachment junction.

17. The device of claim 16, wherein the detachment junction comprises an electrolytically detachable end adapted to detach from a pusher by imposition of a current.

18. A method of occluding a body cavity comprising introducing a vaso-occlusive device according to claim 1 into the body cavity.

19. The method of claim 18, wherein the body cavity is an aneurysm.
